(19) United States
(12) Patent Application Publication
(10) Pub. No.: US 2025/0303108 A1
(43) Pub. Date: Oct. 2, 2025

(54) Apparatus and method for shaping a free end of a tube section

(71) Applicant: Harro Höfliger Verpackungsmaschinen GmbH, Allmersbach im Tal (DE)

(72) Inventors: Bernd POHL, Allmersbach im Tal (DE); Florian SCHAAL, Weissach im Tal (DE); Holger BANTEL, Rudersberg (DE); Jens MARBAZ, Auenwald (DE); Ulrich STAHL, Leutenbach (DE)

(73) Assignee: Harro Höfliger Verpackungsmaschinen GmbH, Allmersbach im Tal (DE)

(21) Appl. No.: 19/090,129

(22) Filed: Mar. 25, 2025

(30) Foreign Application Priority Data
Mar. 27, 2024 (EP) 24 166 677.5

(51) Int. Cl. A61M 25/00

(52) U.S. Cl. CPC A61M 25/001; A61M 2207/00; A61M 2207/10

(57) ABSTRACT

A device for molding a free end of a tube section has a supply unit, a transport unit, and a processing station. The processing station has a tool core, over which the free end of the tube section can be pushed. A molding tool can be positioned around the free end of the tube section, so that the outer contour of the free end can be deformed by the molding tool. The free end and/or the molding tool can be heated by a heating system. In a method, the free end of the tube section is initially introduced into a processing station. The free end is guided over a tool core. Subsequently, the free end and/or the molding tool of the processing station is heated and inserted via a linear movement further into the molding tool, thereby shaping am outer contour of the free end.

### CROSS-REFERENCE TO RELATED APPLICATION

This application claims the benefit of European Patent Application No. EP 24 166 677.5, filed 28-M arch-2024, the content of which is incorporated in its entirety.

### TECHNICAL FIELD

The disclosure relates to a device and a method for molding (shaping) the free end of a tube section. Molding the free end of a tube section may be necessary, in particular, to produce a plastic material cannula with a tip and a head. Plastic material cannulae of this type may be employed, for example, in the subcutaneous administration of medication. In addition, molding the free end of a tube section may also be necessary for further medical applications, for example for urinary catheters, coronary cardiac catheters, central venous catheters or for different suction catheters.

### BACKGROUND

Tube sections or other flexible parts are, as a rule, processed in a plurality of movement steps. It is common practice for the tubes to be initially supplied, cut to length, and picked up and oriented for further processing. Alternatively, it is also possible to use semi-finished products or tube sections which have already been cut to length. The tube sections may be picked up and oriented in particular by means of grippers. EP 3 456 529 B1 discloses a device and a method in which the tube sections can be processed while being transported, so that the tube sections can be processed continuously without having to depart from their orientation.

It is known to thermally deform the free end of a tube section in a corresponding molding tool. Depending on the desired outer contour of the free end of the tube section, it may be necessary, for example, to mold a head separately and subsequently attach it by molding.

### SUMMARY

The disclosure is based on the object of specifying an improved device and an improved method for molding the free end of a tube section, so that it is possible to achieve a fully automated production process, in which particularly precise results can be obtained.

The device for molding the free end of a tube section has a supply unit for the tube section, a transport unit for the tube section and at least one processing station for the free end of the tube section. The processing station has a tool core. The free end of the tube section is guided over this tool core. This has the result that the inner diameter of the tube section remains open. The processing station has a molding tool which can be positioned, from the outside, around the free end of the tube section, so that the outer contour of the free end of the tube section can be deformed by the molding tool. In addition, the processing station has a heating system, by means of which the free end of the tube section and/or the molding tool can be heated up, so that this free end of the tube section can be deformed.

The outer contour of the free end of the tube section after deformation is therefore determined by the inner contour of the molding tool. The quality of the inner contour of the molding tool therefore determines the quality of the outer contour of the deformed tube end. The inner diameter of the deformed tube end is, in contrast, determined by the outer contour of the tool core. The tool core may therefore preferably be designed to be exchangeable, so that it is possible to adapt to different internal diameters of the tube sections in a simple manner. Preferably, the tool core and the molding tool can be exchanged separately from each other so that there is the greatest possible variability with regard to possible contours of the deformed end of the tube section. By employing precision tools, it is therefore possible to achieve a very high level of surface quality of the deformed tube ends.

The tools of the processing station, in particular the molding tool and the tool core, are low-contamination and low-wear tools with a long service life. As a rule, the shaping process can be achieved without the addition of additives, so that the device can also be operated under cleanroom conditions.

The transport unit of the device may preferably enable a linear movement of the tube section to be performed within the processing station, in particular within the molding tool. The free end of the tube section can therefore be displaced within the molding tool during the molding process so that it is also possible, for example, to shape the free end of the tube section inwards or outwards. In this manner, it is possible to mold, for example, a flange directly from the free end of the tube section.

The transport unit may in this case have a gripper unit, with which the tube section to be processed can be fixed. The gripper unit may be fastened to a linear unit so that the gripper unit can be shifted back and forth in a linear movement. Such a linear unit may be, for example, a linear slide block mounted on a rail.

The supply unit may, in principle, vary, depending on the type of tube sections supplied. In a first embodiment, the tube sections, as continuous material, may be reeled off a material roll. The supply unit may in this case have at least one a gripper unit, with which the continuous tube can be progressively reeled off from the material roll. In this case, the supply unit is to have a cutting unit, by means of which the continuous tube can be cut into the tube sections of the desired length. In this case, an upstream processing station may optionally be provided, in which the continuous tube is straightened out. This can be achieved, for example, by straightening the continuous tube.

Alternatively, the tube sections may also be supplied individually or in bulk. In this case, the tube sections may be fastened, for example, manually to the transport unit. The supply unit could in this case also have a separating unit, with which the tube sections are initially separated and oriented so that the latter can be transferred in an automated manner to the transport unit.

Before the tube sections are supplied to the processing station and thus to the molding tool, the tube sections may initially be supplied to at least one upstream processing station. Such an upstream processing station may be, for example, a heat treatment unit and/or an ionization unit. Such an upstream processing station may serve in particular to straighten out the continuous tube reeled off from the material roll. AIternatively or additionally, at least one upstream processing station may have a fan unit to blow any potential particles off the tube sections to be deformed.

Preferably, at least one sensor unit may be provided, by means of which the tube section can be checked. In this manner, the tube section can be checked, for example before shaping, for damage. Such a sensor unit may also be used to inspect the tube sections to be deformed, in particular for irregularities with regard to the internal and external diameters of the tube sections. The deformed tube sections may also be checked with such a sensor unit. In this manner, it is possible to achieve fully automated quality control.

The heating system may preferably operate in accordance with the resistance heating method. The heating process parameters, for example during the heating or cooling phases, may be freely adjusted. Depending on the geometry of the molding tool, the heating zone in the molding tool can be varied. As a rule, heating occurs in the recessed region of the molding tool, i.e. at the site with the smallest cross-sectional area. If the site of the smallest cross-sectional area is relocated to another region of the molding tool, the heating zone can therefore also be relocated.

The processing station may preferably have a cooling means. This cooling means enables the deformed tube end to be cooled particularly swiftly, so that particularly short process times can be achieved.

In a preferred embodiment, the molding tool may have at least two mold parts. The mold parts may be moved towards and away from one another in a defined movement so that the molding tool can be opened and closed. This facilitates the removal of the deformed tube end, in particular in the case of an outwardly or inwardly deformed tube end. This prevents the outwardly and/or inwardly deformed region from being further deformed and thus damaged. The molding tool may be opened in the radial or longitudinal direction, depending on the construction of the individual mold parts. In the event that the molding tool is opened radially, the individual mold parts may, for example, each be designed in the manner of a half-shell and moved apart in a radial manner accordingly. In contrast, in the event that the molding tool is opened in the longitudinal direction, the mold parts may be designed as sleeves which slide into one another.

The device may preferably have a removal device. The removal device enables the tube section with the deformed end to be removed from the transport unit. Subsequently, the tube section can further be cut to length if it is a continuous tube. The tube section which has been cut to size may subsequently be transferred by the removal device to a further station in order to process the tube section further. Such a further station may also be, for example, a further device, in which the opposite free end of the tube section can then be deformed. Such a removal device may be, for example, a robot or an XY gripper.

In the method for molding the free end of a tube section, the free end of the tube section is initially introduced into a processing station via a linear movement. In this process, the free end of the tube section is guided over a tool core of the processing station. The free end of the tube section and/or the molding tool of the processing station is heated. After the desired temperature has been reached, the free end of the tube section is inserted further into the molding tool via a linear movement so that the outer contour of the free end of the tube section can be shaped.

By pushing the free end of the tube section onto the tool core, it is possible to prevent the internal diameter of the tube from melting shut.

Preferably, the tube section may be checked by means of a sensor unit before being introduced into the processing station. It is possible, in particular, to check for possible damage or adhesion points. The external and internal diameters of the tube sections may also be checked by means of the sensor unit. If the tube section to be formed is identified as defective during the check, the tube section may be removed prior to actual processing.

Alternatively or additionally, the tube section may be pretreated by means of an upstream processing station before being introduced into the processing station. The tube section may, for example, be heated up slightly in order to straighten out a continuous tube. Ionization may also be carried out in this context. In addition, particles which may be adhering to the outside could also be blown off the tube section.

The free end of the tube section is introduced into the processing station comprising the molding tool until a predetermined position is reached. This predetermined position may be defined by the distance travelled. Alternatively, the predetermined position may also be defined by the force required to push the free end of the tube section onto the tool core. The respective process parameters for introducing the tube section into the processing station comprising the molding tool are, in principle, freely adjustable. These process parameters may be, for example, the speed during insertion, the acceleration at the beginning of the insertion procedure, the deceleration at the conclusion of the insertion procedure and/or the defined end position.

The process parameters for heating the molding tool may, in principle, be freely adjusted.

After the heating system has been activated, the free end of the tube section is pushed further into the molding tool. The free end of the tube section is shaped by molding, for example, the tip or the head. Depending on the material of the tube section and the type of shaping, the end of the shaping process may be determined by the distance travelled within the molding tool and/or by the force applied to travel within the forming tool and/or by the length of time for travelling within the forming tool.

After the shaping process has ended, the heating of the molding tool of the processing station is terminated by deactivating the corresponding heating system. However, the free end of the tube section may remain in the inserted position for a certain length of time. This produces particularly precise shaping results and minimizes the risk of subsequent, undesirable deformations.

To cool the molding tool more rapidly, a cooling means may be activated after the heating system is deactivated. Cooling may be terminated after the temperature falls below an adjustable temperature threshold and/or after a certain period of time has elapsed.

In an advantageous embodiment, the molding tool may have at least two mold parts so that the molding tool can be opened and closed. In this case, the molding tool is to be closed before the free end of the tube section is inserted further. After the outer contour of the free end of the tube section has been shaped, the molding tool can be reopened in order to facilitate the removal of the deformed tube section. The molding tool is only to be opened after the molding tool has cooled.

The molding tool may be opened, in particular, in a pneumatic or electrical manner. Preferably, one of the mold parts may remain in position so that the deformed end of the tube section may continue to be supported and stabilized by this mold part. The remaining mold parts may then be removed from this stationary mold part.

In order to make it simpler to release the deformed tube section from the molding tool, the molding tool may be heated up again briefly during opening using a defined and controlled heating process.

In order to be able to further process the deformed tube section, the deformed free end of the tube section can be drawn out of the molding tool via a linear movement. As a result, the free end of the tube section is easily accessible and can be removed by a removal device and, for example, transferred to a further processing station.

Such a method may, in principle, be carried out fully automatically, semi-automatically or manually. In a fully automatic process, both the supply of the tube section and the removal of the deformed tube section are performed in an automated manner. In this case, the tube section may further be cut to length before the deformed tube section is actually removed. In this case, the tube sections may be supplied as a continuous tube, wherein the free end can be processed and deformed before the tube section is actually cut to size. If, after the conclusion of the shaping process, the free end of the tube is fixed by the transport unit or the removal device, the tube section can be cut to length, wherein the length of the tube section is, in principle, freely selectable. In a semi-automatic process, it is possible, for example, for the tube sections to be supplied in an automated manner (for example also from a continuous tube), whereas the deformed tube section is removed manually. Alternatively, the tube sections may also be supplied manually by fixing the latter individually to the transport apparatus. In this case, the tube sections may be provided, for example, as bulk material.

The requirements in respect of the shape quality and conformance with exact dimensions of the fully deformed tube sections are, as a rule, very high. The shaping process is highly responsive to changes to individual process parameters, for example with regard to the material of the tube section, temperature, shaping times, speed of the linear movements, molding tools, including the tool cores, and the geometry of the deformed tube sections.

In principle, both ends of a tube section may be processed and deformed using such a device and/or such a method. This allows, for example, the subsequent front end of the tube section to be deformed into a tip, while the subsequent rear end of the tube section may be deformed into a head. Alternatively, both ends of the tube section could also each be deformed into tips, wherein the two tips may in this case be shaped identically or differently. Correspondingly, both ends of the tube section could also be deformed into identical or differently shaped heads. In addition, it would also be possible to process only one of the two ends of a tube section if it is only necessary to deform one of the two ends accordingly.

The shape and geometry (internal and external geometry) of the tip and/or the head is, in principle, freely selectable. There are also no restrictions as regards the length of the tube section in terms of the device and the method.

The method provides a controlled molding process which produces reproducible results. The molding process is position- and/or force-controlled, so that it can be adapted in a simple manner to different requirements.

The device and/or the method can be used to process and shape tube sections made of any material which is, in principle, capable of being shaped.

Further advantages and features of the invention are apparent from the features further specified in the claims and from the following exemplary embodiments.

### BRIEF DESCRIPTION OF THE DRAWINGS

The invention is described and explained in greater detail in the following with reference to the exemplary embodiments illustrated in the drawing, in which:

FIG. 1 shows a schematic view of a first embodiment of the device for molding the free end of a tube section,

FIG. 2 shows a schematic view of the processing station of the device as shown in FIG. 1,

FIG. 3 shows a schematic longitudinal section through the molding tool and the tool tip of the processing station as shown in FIG. 2, and

FIG. 4 shows a schematic longitudinal section through the molding tool and the tool tip of the processing station of a second embodiment of the device.

### DETAILED DESCRIPTION

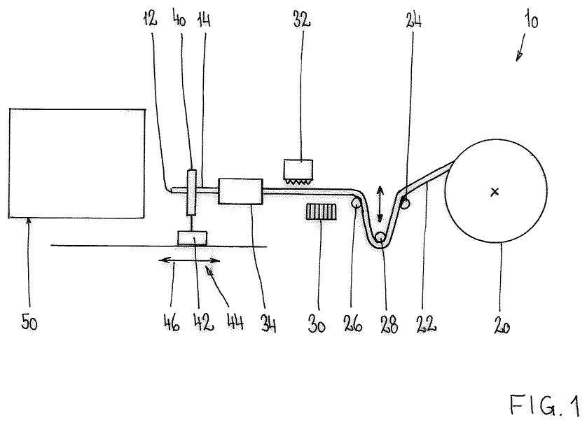

A first embodiment of the device 10 for molding the free end 12 of a tube section 14 is illustrated schematically in FIG. 1.

In the present example, the device 10 has a material roll 20, on which a continuous tube 22 is provided in a reeled manner. The free end 12 of the continuous tube 22 is guided in the present example via two stationary rollers 24, 26 and via a movable roller 28. The movable roller 28 is arranged between the two stationary rollers 24, 26.

The free end 12 is subsequently guided through two upstream processing stations 30, 32. In the upstream processing station 30, the continuous tube 22 is heat-treated; in the upstream processing station 32, the continuous tube 22 is ionized. The two upstream processing stations 30, 32 are intended to reduce the curvature of the continuous tube 22 so as to obtain a continuous tube 22 with the greatest possible degree of straightness. Depending on the material of the continuous tube 22 and the process parameters selected, the processing stations 30, 32 may also be dispensed with.

In addition, it would also be possible to provide further upstream processing stations in which, for example, any potential particles may be blown off the continuous tube 22.

A sensor unit, which may have, for example, a camera system, could be employed before or after the processing stations 30, 32. This allows the continuous tube 22 to be checked for damage and adhesion points. In this manner, it would also be possible to inspect the external and internal diameters of the continuous tube 22.

In the present example, a stationary gripper unit 34 is arranged following the processing stations 30, 32. The gripper unit 34 is not required for reeling off the continuous tube 22 from the material roll 20, so the gripper unit 34 is open while the continuous tube 22 is reeled off. The free end 12 of the continuous tube 22 therefore initially only passes through this stationary gripper unit. A further gripper unit 40 is provided after the stationary gripper unit 34. The gripper unit 40 is fastened to a linear unit 42. The gripper unit 40 is therefore displaceable in a linear manner and, together with the linear unit 42, forms a transport unit 44 for the continuous tube 22. The transport unit 44 enables the continuous tube 22 to be reeled off from the material roll 20. The continuous tube 22 is preferably to be reeled off under uniform tension.

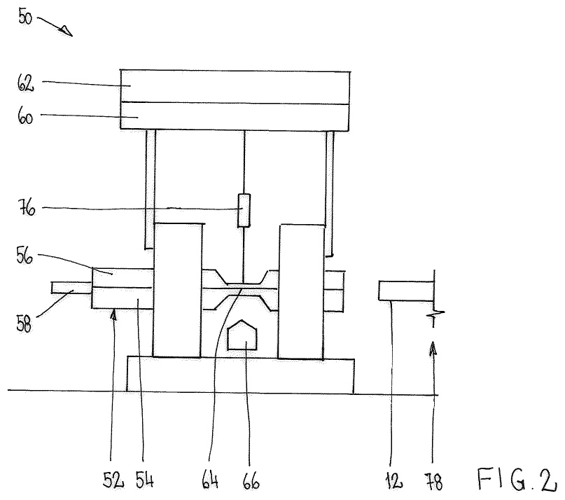

The free end 12 of the continuous tube 22 is held by means of the gripper unit 40. By means of a linear movement (double arrow 46), the free end 12 of the continuous tube 22 can be pushed into a processing station 50 (see in particular FIG. 2). The processing station 50 has a molding tool 52, which in the present example consists of two mold parts 54, 56 in the form of half-shells. In contrast to the exemplary embodiment illustrated here, the molding tool 52 could also be configured in one piece. Alternatively, the molding tool 52 could also have more than two mold parts. The free end 12 of the continuous tube 22 is pushed into the molding tool 52 until a defined position or a defined force is reached. During this movement, the free end 12 of the continuous tube 22 is pushed onto a tool core 58 of the molding tool 52. The diameter of the tool core 58 is adapted to the internal diameter of the continuous tube 22. Preferably, the tool core 58 may be designed as a separate component so that the tool core 58 can be exchanged independently of the molding tool 52. Pushing the free end 12 of the continuous tube 22 onto the tool core 58 prevents the internal diameter of the tube from melting shut during the subsequent shaping process.

The process parameters for pushing the free end 12 into the molding tool 52, in particular the maximum speed, acceleration, deceleration, target position and force applied in the pushing-in procedure are, in principle, freely adjustable. It is thus possible to adapt the process parameters swiftly and simply to different materials and dimensions of the continuous tube 22. It is also possible to adapt to different molding tools 52 without difficulty in this manner.

In addition, the processing station 50 has a heating system 60, via which the molding tool 52 can be heated up directly or indirectly. In the present example, the molding tool 52 is heated up by a continuously controlled current through the molding tool 52. The heating system 60 has an electronic temperature control means 62, so that the heating process parameters, such as, in particular, the predetermined temperatures, the heating ramp, the cooling ramp, the time for which individual temperatures are maintained and further heating control parameters, can be freely adjusted. The molding tool 52 is heated in accordance with the resistance heating principle in the recessed region 64 of the molding tool 52. This recessed region 64 is located in the region with the smallest cross-sectional area of the molding tool 52. By adapting the geometry of the molding tool 52, in particular with regard to the recessed region 64 and the cross-sectional areas of the molding tool 52, the heating zone in the molding tool 52 can be varied and adapted to different circumstances.

After the heating system 60 has been activated, the free end 12 of the continuous tube 22 is pushed further into the molding tool 52 after a defined length of time. In this process, the outer contour 70 of the free end 12 of the continuous tube 22 is shaped. During the shaping process, the outer contour 70 adapts to the inner contour 72 of the molding tool 52. The outer contour 70 of the deformed free end 12 of the continuous tube 22 is therefore defined by the inner contour 72 of the molding tool 52. The inner contour 72 of the forming tool 52 can be manufactured in accordance with specifications, so that, in principle, any desired outer contours 70 can be achieved. The internal diameter of the free end 12 of the molding tool 52 is defined by the selection of the tool core 58. If the external diameter of the tool core 58 is slightly smaller than the internal diameter of the free end 12 of the continuous tube 22, the internal diameter of the continuous tube 22 is reduced accordingly. The length of this reduction in the internal diameter can be determined by the positioning of the tool core 58 within the free end 12.

During the shaping process, the free end 12 can be pushed in in accordance with defined process parameters. These process parameters may be, for example, the acceleration, deceleration and/or feed force. These process parameters can be freely selected depending on the material and the dimensions of the continuous tube 22 and can thus be adapted to the respective circumstances.

The end of the shaping process may be determined by the distance travelled by the transport unit 44 and/or by the force exerted when the continuous tube 22 is pushed into the molding tool 52 and/or by the time taken for the shaping process. After the end of the shaping process, the heating system 60 is deactivated so that the molding tool 52 cools down. In order to accelerate the cooling of the molding tool 52, a cooling means 66 is provided in the present example. This cooling means 66 can be activated after the end of the shaping process. The free end 12 of the continuous tube 22 remains in the inserted position 74 while the molding tool 52 cools down, so that the shaped free end 12 of the continuous tube 22 is not inadvertently damaged or reshaped. This inserted position 74 may be determined, for example, by a distance travelled and/or by a defined feed force.

The temperature of the molding tool 52 may be detected, recorded and evaluated at any time via a temperature measurement means 76. The temperature measurement means 76 may comprise, for example, a resistance measurement means, a thermocouple or a pyrometer. In addition, the temperature measurement means 76 and the temperature control means 62 make it possible to adapt the heating process parameters in a precise manner at any time in the event of non-conformance with the desired temperatures or temperature curves. This makes it possible to respond swiftly to disruptions in the shaping process so that the production of defective non-compliant parts can be reduced.

After the temperature has fallen below a defined temperature and/or after a defined period of time has elapsed, the cooling means is deactivated. The molding tool 52 is subsequently opened. For this purpose, the movable mold part 56 is moved away from the stationary mold part 54 in a linear movement in the radial direction. The deformed free end 12 of the continuous tube 22 remains in the stationary mold part 54, so that the free end 12 continues to be stabilized by the stationary mold part 54. The linear movement of the movable mold part 56 may be driven, in particular, in a pneumatic or electrical manner. In order to facilitate the release of the movable mold part 56 from the deformed free end 12 of the continuous tube 22, the molding tool 52 may be heated up again during opening via a defined and controlled heating process to assist the demolding process.

After the molding tool 52 has been opened, the free end 12 of the continuous tube 22 is drawn out of the stationary mold part 54 by means of the transport unit 44 until the free end 12 is in a removal position 78. In this removal position 78, the free end 12 can be transferred to a removal device, which is not illustrated in greater detail here. Such a removal device may be, for example, a robot arm or an XY gripper.

In the present example, the continuous tube 22 is cut to length before or after being transferred to the removal device 78. The processing station 50 has a cutting apparatus 80 for this purpose. The cutting apparatus 80 enables the continuous tube 22 to be cut into tube sections 14 of any desired length. The length of the tube section 14 is determined in particular by the position of the transport unit 44. The tube is, as a rule, cut to length after the free end 12 of the continuous tube 22 has been shaped, to allow for additional stabilization. Theoretically, however, it would also be possibleto cut the continuous tube 22 to length before shaping. The stationary gripper unit 34 is closed to cut the continuous tube 22 to length, so that the new free end 12, obtained after cutting to length, of the continuous tube 22, is fixed in position and it is not necessary to reorient the free end 12 of the continuous tube 22.

After cutting to length, the finished product can be transferred by the removal device to a further processing station. This may, for example, also be a further device 10, so that the opposite end of the tube section 14 may likewise be shaped.

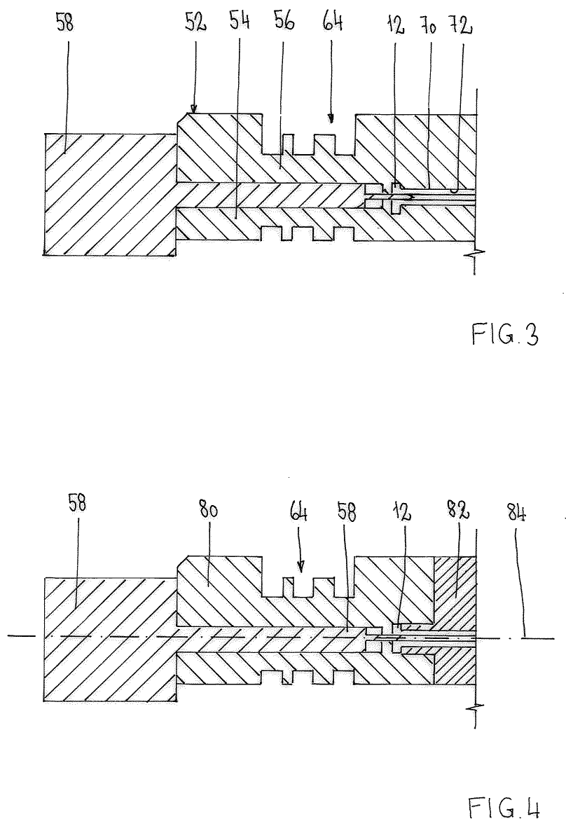

A second embodiment of the molding tool 52.4 is illustrated in FIG. 4. Such a molding tool 52.4 may be employed, in a similar manner to the molding tool 52 shown in FIG. 3, in the processing station 50 of a device 10.

In the present example, the molding tool 52.4 has two sleeve-like mold parts 80, 82. The two mold parts 80, 82 can be slid partially into one another in the longitudinal direction 84. The free end 12 of the continuous tube 22 is pushed onto the tool core 58 of the molding tool 52.4 for shaping, as already described above. The diameter of the tool core 58 is adapted to the internal diameter of the continuous tube 22. Preferably, the tool core 58 may be designed as a separate component so that the tool core 58 can be exchanged independently of the molding tool 52.4.

After the heating system 60 has been activated, the free end 12 of the continuous tube 22 is pushed further into the molding tool 52.4 after a defined length of time. In this process, the outer contour 70 of the free end 12 of the continuous tube 22 is shaped. During the shaping process, the outer contour 70 adapts to the inner contour 72 of the molding tool 52.4. The outer contour 70 of the deformed free end 12 of the continuous tube 22 is therefore defined by the inner contour 72 of the molding tool 52.4. The internal diameter of the free end 12 of the molding tool 52 is defined by the selection of the tool core 58.

After the end of the shaping process, the molding tool 52.4 is opened. For this purpose, the movable mold part 82 is moved out of the stationary mold part 80 in a linear movement in the longitudinal direction. The deformed free end 12 of the continuous tube 22 remains in its shaping position in the region of the stationary mold part 80, so that the free end 12 continues to be stabilized by the stationary mold part 80. The linear movement of the movable mold part 82 may be driven, in particular, in a pneumatic or electrical manner. In order to facilitate the release of the movable mold part 80 from the deformed free end 12 of the continuous tube 22, the molding tool 52.4 may be heated up again during opening via a defined and controlled heating process to assist the demolding process.

The present embodiments are fully automatic processes in which both the supply of the continuous tube 22 and the removal of the fully shaped product are automated.

Alternatively, semi-automatic processes are also possible. In these cases, tube sections 14 which have already been cut to length could be supplied, for example, instead of the continuous tube 22. In this case, the tube section can be fixed in the transport unit 44 in an automated manner or manually by placing the tube sections by hand into the gripper unit 40.

In semi-automatic processes, the finished product can be removed in a semi-automated manner or manually. The finished products could, for example, be removed manually from the stationary mold part 54.

The invention claimed is:

1.-15. (canceled)

16. A device (10) for shaping a free end (12) of a tube section (14, 22), comprising:
a supply unit for the tube section (14, 22);
a transport unit (44) for the tube section (14, 22); and
a processing station (50) for the free end (12) of the tube section (14, 22), the processing station (50) comprising
a tool core (58), configured to receive the free end (12) of the tube section (14, 22) being pushed over the tool core (58),
a molding tool (52, 52.4) configured to be positioned, from outside, around the free end (12) of the tube section (14, 22), whereby an outer contour (70) of the free end (12) of the tube section (14, 22) can be shaped by the molding tool (52, 52.4), and
a heating system (60), for heating a recessed region (64) of the molding tool (52, 52.4) by resistance heating with a continuously controlled current through the molding tool (52, 52.4).

17. The device according to claim 16,
wherein the transport unit (44) enables a linear movement (46) of the tube section (14, 22) to be performed within the molding tool (52, 52.4).

18. The device according to claim 17,
wherein the transport unit (44) has a gripper unit (40) for fixing the tube section (14, 22), and
wherein the gripper unit (40) is fastened to a linear unit (42), whereby the gripper unit (40) can be shifted back and forth in a linear movement (46).

19. The device according to claim 16,
further comprising at least one sensor unit for checking the tube section (14, 22).

20. The device according to claim 16,
wherein the processing station (50) has a cooling means (66).

21. The device according to claim 16,
wherein the molding tool (52, 52.4) has at least two mold parts (54, 56, 80, 82), and
wherein the at least two mold parts (54, 56, 80, 82) can be moved towards and away from one another in a radial direction or in a longitudinal direction (84), in order to close and open the molding tool (52, 52.4).

22. A method for shaping a free end (12) of a tube section (14), comprising:
introducing the free end (12) of the tube section (14, 22) into a processing station (50) via a linear movement (46), and guiding the free end (12) of the tube section (14, 22) over a tool core (58);
resistively heating a molding tool (52, 52.4) of the processing station (50) by continuously controlling a current through the molding tool (52, 52.4) to heat a recessed region (64) of the molding tool (52, 52.4); and
inserting the free end (12) of the tube section (14, 22) further into the molding tool (52, 52.4) via a linear movement (46), and thereby shaping an outer contour (70) of the free end (12) of the tube section (14, 22).

23. The method according to claim 22, further comprising
checking the tube section (14, 22) by at least one sensor unit and/or pretreating the tube section (14, 22) by an upstream processing station (30, 32) before introducing into the processing station (50).

24. The method according to claim 22,
wherein the heating of the molding tool (52, 52.4) is terminated after the shaping has ended, and
wherein the free end (12) of the tube section (14, 22) remains in an inserted position (74) for a predetermined length of time after the shaping has ended and the heating of the molding tool (52, 52.4) has been terminated.

25. The method according to claim 24,
wherein after the heating of the molding tool (52, 52.4) has been terminated, a cooling means (66) is activated.

26. The method according to claim 22, further comprising
closing the molding tool (52, 52.4) before the free end (12) of the tube section (14, 22) is inserted further,
opening the molding tool (52, 52.4) after the outer contour (70) of the free end (12) of the tube section (14, 22) has been shaped in order to facilitate removing the tube section (14, 22).

27. The method according to claim 22, further comprising
heating the molding tool (52, 52.4) during opening.

28. The method according to claim 22, further comprising
removing the free end (12) of the tube section (14, 22) after shaping from the molding tool (52, 52.4) via a linear movement (46).

29. The method according to claim 22, further comprising
supplying a continuous tube (22), and
cutting off the tube section (14) at a desired length from the continuous tube (22) after shaping the outer contour (70) of the free end (12).
